(19) United States
(12) Patent Application Publication
(10) Pub. No.: US 2005/0277127 A1
(43) Pub. Date: Dec. 15, 2005

(54) High-throughput method of DNA immunogen preparation and immunization

(75) Inventors: Maoxin Tian, Burlingame, CA (US); Mark Selby, Burlingame, CA (US); Jang H. Han, Burlingame, CA (US); William J. Rutter, Burlingame, CA (US); Yifel Zhu, Burlingame, CA (US)

Correspondence Address: ROBINS & PASTERNAK, 1731 EMBARCADERO ROAD, SUITE 230, PALO ALTO, CA, 94303, US

(73) Assignee: Epitomics, Inc.

(21) Appl. No.: 10/997,783

(22) Filed: Nov. 23, 2004

Related U.S. Application Data
(60) Provisional application No. 60/525,311, filed on Nov. 26, 2003.
(60) Provisional application No. 60/528,468, filed on Dec. 9, 2003.

(51) Int. Cl. C12Q 1/68; C12P 21/04

(52) U.S. Cl. 435/6

(57) ABSTRACT

A high-throughput process of generating expression-competent, antigen-encoding immunogen DNA through amplification methodology including ligation-assisted PCR is described, as well as the use of the DNA for methods of DNA immunization. Also described is an adjuvant plasmid to enhance antibody production.

This application claims benefit under 35 U.S.C. § 119(e) of provisional application 60/528,468, filed on Dec. 9, 2003, entitled BACTERIAL PLASMID WITH IMMUNOLOGICAL ADJUVANT FUNCTION AND USES THEREOF, and provisional application 60/525,311, filed on Nov. 26, 2003, entitled A HIGH THROUGHPUT METHOD OF DNA IMMUNOGEN PREPARATION AND IMMUNIZATION, all of which applications are hereby incorporated by reference in their entireties.

### TECHNICAL FIELD

The invention relates generally to the field of DNA immunization. In particular, the invention relates to a method of preparing expression-competent DNA as an immunogen and using the immunogen DNA for immunization for antibody production or vaccination. The invention also pertains to the production and use of a plasmid adjuvant for enhancing an immune response to a coadministered immunogen.

### BACKGROUND

Systematic interrogation of the human genome in diseased and normal tissues requires the availability of a high-throughput method of producing antibodies against numerous members of the human proteome. Traditional strategies, which utilize purified antigens to immunize animals do not satisfy the need for high-throughput production.

Plasmid DNA has been used to immunize animals. See, e.g., U.S. Pat. Nos. 5,580,859, 5,589,466 and 6,214,804, all incorporated by reference herein in their entireties. Although this strategy circumvents the need for purifying protein immunogens, it is still not compatible with high-throughput screening since the generation of plasmid DNA requires multiple subcloning steps, transformation and growth of bacteria and preparation of plasmid DNA from the transformed organisms.

Nonsubcloning-based DNA amplification methods, such as PCR, are facile processes amenable to high-throughput modifications. For example, expression-competent DNA can be produced by an overlapping PCR strategy. See, e.g., U.S. Pat. No. 6,280,977, incorporated herein by reference in its entirety. However, this overlapping method is impractical for the high-throughput production of the relatively large amounts of DNA needed for immunization. Thus, there is a need for alternative PCR-based technology in order to generate immunization-sufficient amounts of immunogen DNA.

### SUMMARY OF THE INVENTION

The present invention addresses this need. In particular, the invention provides methods for the rapid and cost-effective preparation of immunogens in the form of amplified DNA, such as PCR DNA, using an automation-amenable set of amplification, restriction and ligation reactions. The DNA made using methods of the invention can be used to induce robust immune responses in animals, such as a humoral and/or cellular immune responses. Thus, the invention is useful for both monoclonal and polyclonal antibody production particularly in cases of high-throughput needs. In addition, the system is useful for vaccination against a wide variety of diseases in animals, including humans. Moreover, the system allows for expression of the immunogen in either an intracellular or extracellular form for maximum antibody responses.

Preparation of immunogen DNA is achieved using a unique ligation-assisted amplification strategy and does not require sequencing confirmation of the product DNA. Alternative methods of DNA amplification can also be utilized. Immunization of animals by the produced immunogen can be carried out using an adjuvant, such as the adjuvant plasmid described herein. The invention finds application in the fields of molecular biology and immunology, diagnosis and therapy of cancer and immune diseases.

The invention also relates to a novel adjuvant plasmid for use in immunogenic compositions, such as in compositions with the immunogen DNA described above, as well as in vaccine compositions containing protein and/or other DNA immunogens.

These and other embodiments of the subject invention will readily occur to those of skill in the art in view of the disclosure herein.

### BRIEF DESCRIPTION OF THE FIGURES

FIG. 1 shows a representative strategy for generating vector DNA to be used in the subject methods. A polynucleotide sequence encoding a protein immunogen is amplified using a forward primer (SEQ ID NO:1) and a reverse primer (SEQ ID NO:2) that contain restriction sites for the endonuclease, Sfi1. The amplified polynucleotide is digested with Sfi1 and ligated into a vector cut with Sfi1. The vector contains control elements that direct expression of the immunogen, including a CMV promoter, an Ig-κ secretion signal, and a polyadenylation sequence. The vector, containing the immunogen-encoding insert, is amplified to produce DNA for immunization.

FIG. 2 is a representation of the final amplified MTF product using a combined ligation/amplification method as detailed herein.

FIG. 3 is a representation of the final amplified products of 10 genes using a combined ligation/amplification method as detailed herein.

FIG. 4 is a representation of an immunoblot showing the production of HA-CDC42 using the method of the invention.

FIG. 5 is a diagrammatic representation of an adjuvant plasmid for use with the present invention.

FIG. 6 shows the results of a nucleic acid immunization experiment using the constructs of the invention. A=animals administered adjuvant plasmid (encoding) containing a cytokine-encoding segment consisting of SLC-IL4 fusion, IRES and CD40 ligand under the transcriptional control of the EF1alpha promoter; B=animals administered the adjuvant plasmid's non-coding variant containing the cytokine segment in the opposite orientation; C=animals given an empty plasmid lacking the promoter and the cytokine segment; D=animals immunized with PSA without any plasmid DNA; E=animals given a control immunization.

FIG. 7 depicts the results of experiments using the secretory signals TAT and Ig-kappa, as described in the examples.

FIGS. 8A-8B depict the sequence of a plasmid SlcIl4IresCD40LpORF (SEQ ID NO:3). The SLC gene spans nucleotide positions 690 to 1088; the IL-4 gene spans nucleotide positions 1090 to 1452; the SLC-IL-4 fusion spans nucleotide positions 690 to 1452; the IRES spans nucleotide positions 1459 to 2007; the CD40 ligand sequence spans positions 2010-2792.

FIGS. 9A-9B depict the sequence of the non-coding version of plasmid SlcIl4IresCD40LpORF (SEQ ID NO:4). The segment from nucleotide position 690 to nucleotide position 2007 of SEQ ID NO:3, which contains the SLC-IL-4 fusion and IRES sequences, is reversed in non-coding orientation.

### DETAILED DESCRIPTION OF THE INVENTION

The practice of the present invention will employ, unless otherwise indicated, conventional methods of molecular biology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

1. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a coding sequence” includes a mixture of two or more coding sequences, and the like.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

The terms “analog” and “mutein” refer to biologically active derivatives of the reference molecule, or fragments of such derivatives, that retain desired activity, such as immunoreactivity in the assays described herein. In general, the term “analog” refers to compounds having a native polypeptide sequence and structure with one or more amino acid additions, substitutions (generally conservative in nature, or in the case of a modified MEFA, generally non-conservative in nature at the NS3 proteolytic cleavage sites) and/or deletions, relative to the native molecule, so long as the modifications do not destroy immunogenic activity. The term “mutein” refers to polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem Biol. (2000) 7:463-473; and Simon et al., Proc. Natl. Acad. Sci. USA (1992) 89:9367-9371 for descriptions of peptoids). Preferably, the analog or mutein has at least the same immunoactivity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “fragment” is intended a polypeptide consisting of only a part of the intact full-length polypeptide sequence and structure. The fragment can include a C-terminal deletion an N-terminal deletion, and/or an internal deletion of the native polypeptide.

An “antigen” refers to a molecule, such as a polypeptide as defined above, containing one or more epitopes (either linear, conformational or both) that will stimulate a host's immune system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. Similarly, an oligonucleotide or polynucleotide that expresses an antigen or antigenic determinant in vivo, such as in nucleic acid immunization applications, is also included in the definition of antigen herein. For purposes of the present invention, immunogens can be derived from any organism for which an immune response is desired, including immunogens derived from viruses, bacteria, fungi, parasites and the like.

By “immunogenic fragment” is meant a fragment of the reference polypeptide that includes one or more epitopes and thus elicits one or more of the immunological responses described herein. An “immunogenic fragment” of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains the ability to elicit an immunological response as defined herein.

The term “epitope” as used herein refers to a sequence of at least about 3 to 5, preferably about 5 to 10 or 15, and not more than about 500 amino acids (or any integer therebetween), which define a sequence that by itself or as part of a larger sequence, elicits an immunological response in the subject to which it is administered. Often, an epitope will bind to an antibody generated in response to such sequence. There is no critical upper limit to the length of the epitope, which may comprise nearly the full-length of the protein sequence, or even a fusion protein comprising two or more epitopes from the molecule in question. An epitope for use in the subject invention is not limited to a polypeptide having the exact sequence of the portion of the parent protein from which it is derived. For example, viral genomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1985) Proc. Natl. Acad. Sci. USA 82:178-182; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

The ability of a particular immunogen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Recent methods of measuring cell-mediated immune response include measurement of intracellular cytokines or cytokine secretion by T-cell populations, or by measurement of epitope specific T-cells (e.g., by the tetramer technique) (reviewed by McMichael, A. J., and O'Callaghan, C. A., J. Exp. Med. (1998) 187:1367-1371; Mcheyzer-Williams et al, Immunol. Rev. (1996) 150:5-21; Lalvani et al., J. Exp. Med. (1997) 186:859-865.

Thus, an immunological response as used herein may be one that stimulates the production of antibodies (e.g., neutralizing antibodies that block viruses from entering cells and/or replicating by binding to the pathogens, typically protecting cells from infection and destruction). The antigen of interest may also elicit production of CTLs. Hence, an immunological response may include one or more of the following effects: the production of antibodies by B-cells; and/or the activation of suppressor T-cells and/or δγ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art. (See, e.g., Montefiori et al., J. Clin Microbiol. (1988) 26:231-235; Dreyer et al., AIDS Res Hum Retroviruses (1999) 15:1563-1571).

An “immunogenic composition” is a composition that comprises an antigenic molecule, such as a DNA fragment according to the invention, where administration of the composition to a subject results in the development in the subject of a humoral and/or a cellular immune response to the antigenic molecule of interest. The immunogenic composition can be introduced directly into a recipient subject, such as by injection, inhalation, oral, intranasal and mucosal (e.g., intrarectally or intravaginally) administration.

An adjuvant composition comprising an adjuvant plasmid as described herein “enhances” or “increases” the immune response, or displays “enhanced” or “increased” immunogenicity vis-a-vis a selected antigen when it possesses a greater capacity to elicit an immune response than the immune response elicited by an equivalent amount of the antigen when delivered without the adjuvant plasmid. Such enhanced immunogenicity can be determined by administering the antigen and adjuvant, and antigen controls to animals and comparing antibody titers against the two using standard assays such as radioimmunoassay and ELISAs, well known in the art.

“Homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, DC, which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “coding sequence” or a sequence which “encodes” a selected polypeptide, is a nucleic acid molecule which is transcribed and translated into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

“Operably linked” refers to an arrangement of elements wherein the components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can transcribed introns, and the promoter sequence can still be considered “operably linked” to the coding sequence.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

A “control element” refers to a polynucleotide sequence which aids in the expression of a coding sequence to which it is linked. The term includes promoters, transcription termination sequences, upstream regulatory domains, polyadenylation signals, untranslated regions, including 5′-UTRs and 3′-UTRs and when appropriate, leader sequences and enhancers, which collectively provide for the transcription and translation of a coding sequence in a host cell.

A “promoter” as used herein is a regulatory region capable of binding RNA polymerase in a host cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

A control sequence “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest. The expression cassette includes control elements, as described above, such as a promoter which is operably linked to (so as to direct transcription of) the sequence(s) or gene(s) of interest, and often includes a polyadenylation sequence as well. As used herein, the terms “expression cassette” or “expression construct” do not necessarily imply that the cassette or construct is present in a plasmid.

The term “infrequently cutting restriction endonuclease” refers to a restriction endonuclease which cuts at sites that occur infrequently in a polynucleotide sequence because of having a relatively long recognition sequence. Many such infrequently cutting restriction endonucleases are known, see Sambrook et al., supra. Infrequently cutting restriction endonucleases include, but are not limited to, Sfi1, BstAP1, PfiM1, Mwo1, AlwN1, NotI, SalI, and MluI.

By “nucleic acid immunization” is meant the introduction of a nucleic acid molecule encoding one or more selected immunogens into a host cell, for the in vivo expression of the immunogen. The nucleic acid molecule can be introduced directly into a recipient subject, such as by injection, inhalation, oral, intranasal and mucosal administration, or the like, or can be introduced ex vivo, into cells which have been removed from the host. In the latter case, the transformed cells are reintroduced into the subject where an immune response can be mounted against the immunogen encoded by the nucleic acid molecule.

A “DNA-dependent DNA polymerase” is an enzyme that synthesizes a complementary DNA copy from a DNA template. Examples are DNA polymerase I from E. coli and bacteriophage T7 DNA polymerase. All known DNA-dependent DNA polymerases require a complementary primer to initiate synthesis. Under suitable conditions, a DNA-dependent DNA polymerase may synthesize a complementary DNA copy from an RNA template.

A “DNA-dependent RNA polymerase” or a “transcriptase” is an enzyme that synthesizes multiple RNA copies from a double-stranded or partially-double stranded DNA molecule having a (usually double-stranded) promoter sequence. The RNA molecules (“transcripts”) are synthesized in the 5′ to 3′ direction beginning at a specific position just downstream of the promoter. Examples of transcriptases are the DNA-dependent RNA polymerase from E. coli and bacteriophages T7, T3, and SP6.

An “RNA-dependent DNA polymerase” or “reverse transcriptase” is an enzyme that synthesizes a complementary DNA copy from an RNA template. All known reverse transcriptases also have the ability to make a complementary DNA copy from a DNA template; thus, they are both RNA- and DNA-dependent DNA polymerases. A primer is required to initiate synthesis with both RNA and DNA templates.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The “target sequence” may also include the complexing sequences to which the oligonucleotide primers complex and extend using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands.

The term “primer” or “oligonucleotide primer” as used herein, refers to an oligonucleotide which acts to initiate synthesis of a complementary nucleic acid strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

The “melting temperature” or “Tm” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25 degrees C. below the Tₘ. The Tₘ may be estimated using the following relationship: Tₘ=69.3+0.41 (GC)% (Marmur et al. (1962) J. Mol. Biol. 5:109-118).

The terms “effective amount” or “pharmaceutically effective amount” of an immunogenic composition, as provided herein, refer to a nontoxic but sufficient amount of the composition to provide the desired response, such as an immunological response, and optionally, a corresponding therapeutic effect. The exact amount required will vary from subject to subject, depending on the species, age, and general condition of the subject, the severity of the condition being treated, and the particular macromolecule of interest, mode of administration, and the like. An appropriate “effective” amount in any individual case may be determined by one of ordinary skill in the art using routine experimentation.

The term “treatment” as used herein refers to either (1) the prevention of infection or reinfection (prophylaxis), or (2) the reduction or elimination of symptoms of the disease of interest (therapy).

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species, since the immune systems of all of these vertebrates operate similarly.

2. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of a rapid and high-throughput methodology of antibody production. In the practice of the present invention, an immunogen is generated in a DNA form that is not a plasmid, through a novel ligation/amplification technology. The non-plasmid DNA can be introduced into the subject using any of various DNA delivery techniques, described more fully below. Additionally, adjuvants and the like can be used to enhance the immune response elicited by the protein encoded by the delivered DNA. The DNA fragments can also be used to produce antibodies, both polyclonal and monoclonal, for use as diagnostics, immunopurification reagents and the like.

This technique of immunogen generation has several advantages over previous methods. In particular, the techniques described herein allow immunogen production in a high throughput and time-saving manner. Moreover, the methods provide the ability to immunize animals in cases in which protein forms of the antigens are difficult to produce. Additionally, the methods facilitate selection of a desired region from the whole protein/open reading frame for epitope-specific antibody generation. Furthermore, the antigen can be expressed in either an intracellular or extracellular form for maximum antibody responses.

Specifically, the immunizing DNA is generated through the combination of (a) an amplification step, such as polymerase chain reaction in which the complete or a selected region of a target gene is amplified; (b) preparation of DNA fragments that can provide regulatory segments (e.g., promoter and polyA signals), in either a prepared vector fragment form, a plasmid or two amplification products of the two segments; (c) linkage of the antigen sequence to the regulatory segments via restriction digestion and ligation; and (d) final amplification of the transcriptionally and translationally competent constructs including, for example, three segments in the configuration of [promoter]-[protein-encoding sequence]-[polyA].

Polynucleotides coding for immunogens for use with the present invention can be derived from a wide variety of organisms, such as bacteria, viruses, fungi and parasites and can be obtained using standard techniques. Polynucleotide sequences coding for the above-described molecules are generally obtained by screening cDNA and/or genomic libraries from cells expressing the gene, or by deriving the gene from a vector known to include the same. For example, polynucleotides encoding the immunogenic polypeptides of interest can be isolated from a genomic library derived from nucleic acid sequences present in, for example, the plasma, serum, or tissue homogenate of an infected individual.

In particular, the present invention makes use of forward (sense) and reverse (antisense) primers that contain unique restriction sites to facilitate ligation of the amplified coding sequence of interest into vector DNA. The vector DNA includes control sequences for driving transcription of the coding sequence in a vertebrate host in vivo. The restriction sites can be sites for use with any of the well known restriction enzymes. Restriction enzymes with various specificities have been isolated from a wide range of prokaryotes and are well known in the art. See, e.g., Sambrook et al., supra. The choice of an appropriate restriction site is largely a matter of choice. The two restriction sites when digested by the appropriate restriction enzyme should yield overhang ends with different sequences. One of skill in the art will readily recognize the proper restriction sites and enzymes to use for a desired sequence. As detailed in the examples, one particularly preferred restriction enzyme is Sfi1.

Generally, the primers will include about 4 to about 50 nucleotides, more preferably about 5 to about 25 nucleotides, and most preferably about 7 to about 15 nucleotides, such as 8, 9, 10, 11, 12, 13, 14 . . . nucleotides.

The primers are then used in amplification reactions, to obtain the protein-encoding sequence. Particularly useful amplification techniques include polymerase chain reaction (PCR)-based techniques, such as PCR and RT-PCR. PCR is a technique for amplifying a desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers as described above which flank the target nucleotide sequence to be amplified, oriented such that their 3′ ends face each other, each primer extending toward the other. The polynucleotide sample is extracted and denatured, preferably by heat, and hybridized with first and second primers that are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. Preferably, PCR is carried out with a commercially available thermal cycler, e.g., Perkin Elmer.

RNAs may be amplified by reverse transcribing the RNA into cDNA, and then performing PCR (RT-PCR), as described above. Alternatively, a single enzyme may be used for both steps as described in U.S. Pat. No. 5,322,770, incorporated herein by reference in its entirety. RNA may also be reverse transcribed into cDNA, followed by asymmetric gap ligase chain reaction (RT-AGLCR) as described by Marshall et al. (1994) PCR Meth. App. 4:80-84.

The Ligase Chain Reaction (LCR) is an alternate method for nucleic acid amplification. In LCR, probe pairs are used which include two primary (first and second) and two secondary (third and fourth) probes, all of which are employed in molar excess to target. The first probe hybridizes to a first segment of the target strand, and the second probe hybridizes to a second segment of the target strand, the first and second segments being contiguous so that the primary probes abut one another in 5′ phosphate-3′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. If the target is initially double stranded, the secondary probes also will hybridize to the target complement in the first instance. Once the ligated strand of primary probes is separated from the target strand, it will hybridize with the third and fourth probes which can be ligated to form a complementary, secondary ligated product. It is important to realize that the ligated products are functionally equivalent to either the target or its complement. By repeated cycles of hybridization and ligation, amplification of the target sequence is achieved. This technique is described more completely in EPA 320,308 to K. Backman published Jun. 16, 1989 and EPA 439,182 to K. Backman et al., published Jul. 31, 1991, both of which are incorporated herein by reference.

Other known amplification methods which can be utilized herein include but are not limited to the so-called “NASBA” or “3SR” technique described by Guatelli et al., Proc. Natl. Acad. Sci. USA (1990) 87:1874-1878 and J. Compton, Nature (1991) 350:91-92 (1991); Q-beta amplification; strand displacement amplification (as described in Walker et al., Clin. Chem. (1996) 42:9-13 and EPA 684,315; and target mediated amplification, as described in International Publication No. WO 93/22461.

Once amplified, the protein-encoding sequence will contain the restriction sites described above. This construct is then ligated into a vector, such as a non-plasmid vector fragment, which contains appropriate control and regulatory sequences such that the coding sequence can be transcribed in vivo to produce the immunogen which in turn elicits an immure response. In order to facilitate appropriately ordered ligation of the DNA fragment bearing the gene of interest with the vector, the vector also contains the same restriction sites that flank the coding sequence such that treatment with the appropriate enzyme will produce cohesive termini complimentary to the 3′ and 5′ termini of the control sequences in the vector fragment. In this way, ordered association of the DNA fragment and the vector occurs.

The promoter for use in the vector fragment is one which will direct transcription of the gene of interest in a vertebrate subject when the gene of interest is operably linked thereto. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′- UTR sequences can be placed adjacent to the coding sequence in order to enhance expression of the same. Such sequences include UTRs which include an Internal Ribosome Entry Site (IRES) present in the leader sequences of picornaviruses such as the encephalomyocarditis virus (EMCV) UTR (Jang et al. J. Virol. (1989) 63:1651 -1660. Other picornavirus UTR sequences that will also find use in the present invention include the polio leader sequence and hepatitis A virus leader and the hepatitis C IRES.

Moreover, a sequence encoding a signal peptide or leader sequence may be present. Indeed, as shown in the examples below, such sequences can enhance the antibody response by transporting intracellular protein outside of the cell to provide greater access to the immune system. If a signal sequence is included, it can either be the native, homologous sequence, or a heterologous sequence. A variety of suitable signal sequences are known including, without limitation, the yeast invertase gene (EPO Publication No. 012,873; JPO Publication No. 62,096,086), various α-factor leaders (U.S Pat. Nos. 4,546,083 and 4,870,008), the interferon leader (EPO Publication No. 060,057), the adenovirus tripartite leader, the tpa leader, the tat sequence, the Ig-kappa secretory sequence, and the like.

The DNA fragment and the vector fragment which has been digested with the particular restriction enzyme of interest are then ligated together using a DNA ligase and techniques well known in the art to produce an expression vector, with the coding sequence and control sequences positioned and oriented such that the coding sequence is transcribed under the “control” of the control sequences (i.e., RNA polymerase which binds to the DNA molecule at the control sequences transcribes the coding sequence). Once produced, the expression vector is than amplified using any suitable amplification technique such as any of the techniques described above. The DNA immunogens can then be isolated using any of several known techniques.

Compositions and Administration

The invention provides compositions including the above-described immunogen DNA. Such compositions can be formulated as injectables, either as liquid solutions or suspensions; solid forms suitable for solution in, or suspension in, liquid vehicles prior to injection may also be prepared. The compositions generally include excipients, such as water, saline, glycerol, dextrose, ethanol, or the like, singly or in combination, as well as substances such as wetting agents, emulsifying agents, or pH buffering agents.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art.

If desired, co-stimulatory molecules which improve immunogen presentation to lymphocytes, such as B7-1 or B7-2, or cytokines such as GM-CSF, IL-2, and IL-12, can be included in a composition of the invention.

Optionally, adjuvants can also be coadministered with the DNA immunogens, either in the same or different compositions. If administered separately, the adjuvant can be given concurrently, prior to, or subsequent to the DNA immunization. If administered prior to immunization with the DNA immunogen, the adjuvant formulations can be administered as early as 5-10 days prior to immunization, preferably 3-5 days prior to immunization and most preferably 1-3 or 2 days prior to immunization with the DNA immunogen of interest. If administered separately, the adjuvant formulation can be delivered either to the same site of delivery as the DNA immunogen composition or to a different delivery site.

If simultaneous delivery is desired, the DNA immunogen can be included with the adjuvant. Generally, the DNA immunogen and adjuvant can be combined by simple mixing, stirring, or shaking. Other techniques, such as passing a mixture of the two components rapidly through a small opening (such as a hypodermic needle) can also be used to provide the vaccine compositions.

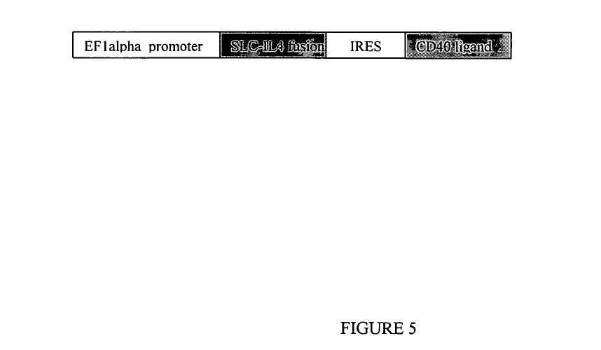

Adjuvants which can be used include, but are not limited to: (1) aluminum salts (alum), such as aluminum hydroxide, aluminum phosphate, aluminum sulfate, etc.; (2) oil-in-water emulsion formulations; (3) saponin adjuvants; (4) Complete Freund's Adjuvant (CFA) and Incomplete Freund's Adjuvant (IFA); (5) cytokines, such as interleukins (IL-1, IL-2, IL-4, IL-5, IL-6, IL-7, IL-12, interferons, macrophage colony stimulating factor (M-CSF), tumor necrosis factor (TNF), etc.; (6) detoxified mutants of a bacterial ADP-ribosylating toxin such as a cholera toxin (CT), pertussis toxin (PT), or an E. coli heat-labile toxin (LT); (7) oligonucleotides comprising CpG motifs (See, e.g., U.S. Pat. No. 6,207,646; Krieg et al. Nature (1995) 374:546 and Davis et al. J. Immunol. (1998) 160:870-876); as well as other immunostimulatory molecules. A particularly desirable adjuvant for use in the present methods, as well as for use with other immunogens, is the plasmid adjuvant termed SlcIl4IresCD40LpORF (see, FIG. 5 and the examples herein; and commonly owned, copending U.S. patent application entitled “Bacterial Plasmid with Immunological Adjuvant Function and Uses Thereof” (Attorney docket number 7037-0002), filed Nov. 26, 2004, incorporated herein by reference in its entirety).

Additionally, the expression constructs can be packaged in liposomes prior to delivery to the cells. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527.

Compositions for use in the invention will comprise a therapeutically effective amount of the desired DNA molecule and any other of the above-mentioned components, as needed. By “therapeutically effective amount” is meant an amount of DNA immunogen which will induce an immunological response, either for antibody production or for treatment or prevention of a particular disease or infection. Such a response will generally result in the development in the subject of an antibody-mediated and/or a secretory or cellular immune response to the composition. Usually, such a response includes but is not limited to one or more of the following effects; the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell, and/or cytotoxic T cell and/or γδT cell populations.

Once formulated, the compositions are conventionally administered parenterally, e.g., by injection, either subcutaneously, intraperitoneally, intramuscularly or intravenously. Additional formulations suitable for other modes of administration include oral and pulmonary formulations, suppositories, and transdermal formulations, aerosol, intranasal, and sustained release formulations.

Dosage treatment may be a single dose schedule or a multiple dose schedule. The exact amount necessary will vary depending on the desired response, i.e., antibody production and/or a protective immune response; the subject being treated; the age and general condition of the individual to be treated; the capacity of the individual's immune system to synthesize antibodies; the degree of protection desired; the severity of the condition being treated; the particular macromolecule selected and its mode of administration, among other factors. An appropriate effective amount can be readily determined by one of skill in the art. A “therapeutically effective amount” will fall in a relatively broad range that can be determined through routine trials using in vitro and in vivo models known in the art.

The compositions can be delivered using standard gene delivery protocols. Methods for gene delivery are known in the art. See, e.g., U.S. Pat. Nos. 5,399,346, 5,580,859, 5,589,466, incorporated by reference herein in their entireties. Genes can be delivered either directly to the subject or, alternatively, delivered ex vivo, to cells derived from the subject and the cells reimplanted in the subject.

A wide variety of methods can be used to deliver the expression constructs to cells. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, liposomes, peptoid delivery, or microinjection. See, e.g., Sambrook et al., supra, for a discussion of techniques for transforming cells of interest; and Felgner, P. L., Advanced Drug Delivery Reviews (1990) 5:163-187, for a review of delivery systems useful for gene transfer. Methods of delivering DNA using electroporation are described in, e.g., Selby et al., J. Immunol. (2000) 164:4635-4640; U.S. Pat. Nos. 6,132,419; 6,451,002, 6,418,341, 6233,483, U.S. Patent Publication No. 2002/0146831; and International Publication No. WO/0045823, all of which are incorporated herein by reference in their entireties.

Additionally, biolistic delivery systems employing particulate carriers such as gold and tungsten, are useful for delivering the expression constructs of the present invention. The particles are coated with the construct to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744.

The amount of DNA delivered will generally be about 1 μg to 500 mg of DNA, such as 5 μg to 100 mg of DNA, e.g., 10 μg to 50 mg, or 100 μg to 5 mg, such as 20 . . . 30 . . . 40 . . . 50 . . . 60 . . . 100 . . . 200 μg and so on, to 500 μg DNA, and any integer between the stated ranges.

Administration of the polynucleotide compositions can elicit an antibody titer and/or a cellular immune response in the animal that lasts for at least 1 week, 2 weeks, 1 month, 2 months, 3 months, 4 months, 6 months, 1 year, or longer. The compositions can also be administered to provide a memory response. If such a response is achieved, antibody titers may decline over time, however exposure to the particular immunogen results in the rapid induction of antibodies, e.g., within only a few days. Optionally, antibody titers can be maintained in a subject by providing one or more booster injections of the compositions, at e.g., 2 weeks, 1 month, 2 months, 3 months, 4 months, 5 months, 6 months, 1 year, or more after the primary injection.

Preferably, an antibody titer of at least 10, 100, 150, 175, 200, 300, 400, 500, 750, 1,000, 1,500, 2,000, 3,000, 5,000, 10,000, 20,000, 30,000, 40,000, 50,000 (geometric mean titer), or higher, is elicited, or any number between the stated titers, as determined using a standard immunoassay.

Antibodies

The DNA immunogens can be used to produce immunogen-specific polyclonal and monoclonal antibodies. Such specific polyclonal and monoclonal antibodies specifically bind to the immunogen in question. Polyclonal antibodies can be produced by administering the immunogen to a mammal, such as a mouse, a rabbit, a goat, or a horse. Serum from the immunized animal is collected and the antibodies are purified from the plasma by, for example, precipitation with ammonium sulfate, followed by chromatography, preferably affinity chromatography. Techniques for producing and processing polyclonal antisera are known in the art.

Monoclonal antibodies directed against specific epitopes encoded by the DNA immunogen can also be readily produced. Normal B cells from a mammal, such as a mouse, immunized with a DNA immunogen, can be fused with, for example, HAT-sensitive mouse myeloma cells to produce hybridomas. Hybridomas producing specific antibodies can be identified using RIA or ELISA and isolated by cloning in semi-solid agar or by limiting dilution. Clones producing the specific antibodies in question are isolated by another round of screening.

It may be desirable to provide chimeric antibodies, especially if the antibodies are to be used in preventive or therapeutic pharmaceutical preparations, such as for providing passive protection against infection, as well as in diagnostic preparations. Chimeric antibodies composed of human and non-human amino acid sequences may be formed from the mouse monoclonal antibody molecules to reduce their immunogenicity in humans (Winter et al. (1991) Nature 349:293; Lobuglio et al. (1989) Proc. Nat. Acad. Sci. USA 86:4220; Shaw et al. (1987) J. Immunol. 138:4534; and Brown et al. (1987) Cancer Res. 47:3577; Riechmann et al. (1988) Nature 332:323; Verhoeyen et al. (1988) Science 239:1534; and Jones et al. (1986) Nature 321:522; EP Publication No. 519,596, published 23 Dec. 1992; and U.K. Patent Publication No. GB 2,276,169, published 21 Sep. 1994).

Antibody molecule fragments, e.g., F(ab′)₂, Fv, and sFv molecules, that are capable of exhibiting immunological binding properties of the parent monoclonal antibody molecule can be produced using known techniques. Inbar et al. (1972) Proc. Nat. Acad. Sci. USA 69:2659; Hochman et al. (1976) Biochem 15:2706; Ehrlich et al. (1980) Biochem 19:4091; Huston et al. (1988) Proc. Nat. Acad. Sci. USA 85(16):5879; and U.S. Pat. Nos. 5,091,513 and 5,132,405, to Huston et al.; and 4,946,778, to Ladner et al.

In the alternative, a phage-display system can be used to expand monoclonal antibody molecule populations in vitro. Saiki, et al. (1986) Nature 324:163; Scharf et al. (1986) Science 233:1076; U.S. Pat. Nos. 4,683,195 and 4,683,202; Yang et al. (1995) J Mol Biol 254:392; Barbas, III et al. (1995) Methods: Comp. Meth Enzymol 8:94; Barbas, III et al. (1991) Proc Natl Acad Sci USA 88:7978.

Once generated, the phage display library can be used to improve the immunological binding affinity of the Fab molecules using known techniques. See, e.g., Figini et al. (1994) J. Mol. Biol. 239:68. The coding sequences for the heavy and light chain portions of the Fab molecules selected from the phage display library can be isolated or synthesized, and cloned into any suitable vector or replicon for expression. Any suitable expression system can be used, including any of the various expression systems known in the art.

Antibodies which are directed against epitopes from a particular pathogen, are particularly useful for detecting the presence of that pathogen in a sample, such as a serum sample from an individual suspected of infection. An immunoassay may utilize one antibody or several antibodies. An immunoassay may use, for example, a monoclonal antibody directed towards a particular epitope, a combination of monoclonal antibodies directed towards multiple epitopes of a single pathogen, monoclonal antibodies directed towards epitopes of different pathogens, polyclonal antibodies directed towards the same pathogen, polyclonal antibodies directed towards different pathogens, or a combination of monoclonal and polyclonal antibodies. Immunoassay protocols may be based, for example, upon competition, direct reaction, or sandwich type assays using, for example, labeled antibody. The labels may be, for example, fluorescent, chemiluminescent, or radioactive.

The antibodies generated may also be used to isolate pathogens or antigens by immunoaffinity columns. The antibodies can be affixed to a solid support by, for example, adsorption or by covalent linkage so that the antibodies retain their immunoselective activity. Optionally, spacer groups may be included so that the antigen binding site of the antibody remains accessible. The immobilized antibodies can then be used to bind pathogens or antigens from a biological sample, such as blood or plasma. The bound substances are recovered from the column matrix by, for example, a change in pH.

3. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

Enzymes were purchased from commercial sources, and used according to the manufacturers' directions.

In the isolation of DNA fragments, except where noted, and all DNA manipulations were done according to standard procedures. See, e.g., Sambrook et al., supra. Restriction enzymes, T₄ DNA ligase, DNA polymerase 1I, Klenow fragment, and other biological reagents can be purchased from commercial suppliers and used according to the manufacturers' directions. Sources for chemical reagents generally include Sigma Chemical Company, St. Louis, Mo.; Alrich, Milwaukee, Wis.; Roche Molecular Biochemicals, Indianapolis, Ind.

### EXAMPLE 1

### Generation of High Quantities of Expression-Competent PCR Constructs

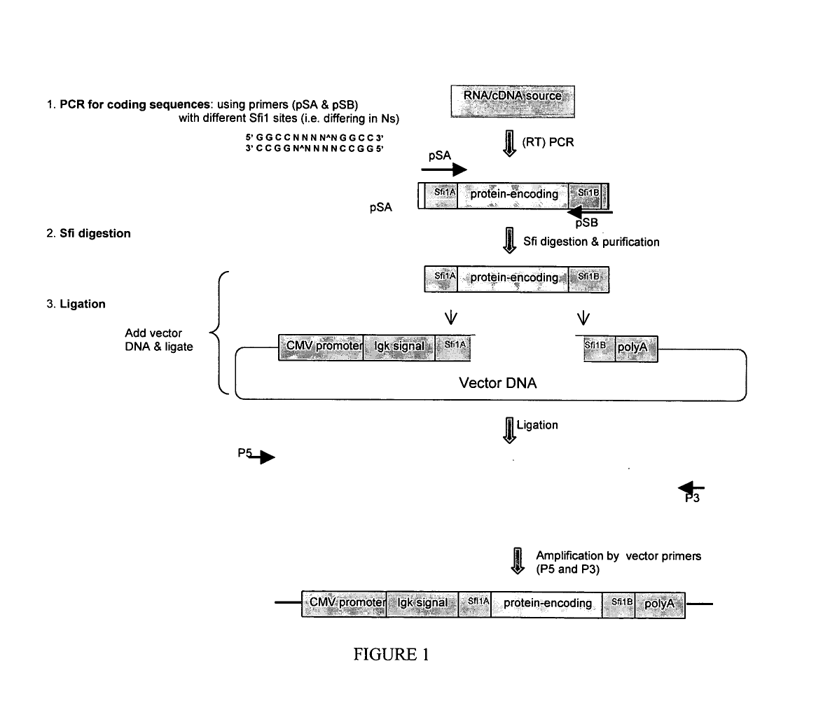

DNA constructs were prepared as diagrammed in FIG. 1 as follows.

1A. Designing PCR Primers with Special Restriction Sites

A unique restriction site in each of the two primers that were to be used to amplify the protein-encoding region of choice was provided. The two restriction sites, upon digestion with the corresponding restriction enzymes, yielded overhang ends with different sequences.

For this purpose, the Sfi1 restriction enzyme was a convenient choice, however other restriction enzymes will also find use with the methods. Two Sfi1-recognizable sequences (such as GGCCATGAAGGCC (SEQ ID NO:1) and GGCCGAGGCGGCC (SEQ ID NO:2)), differing in the middle in a 5 basepair region (underlined) flanked by the Sfi1 recognition sequences, was built into the two primers (pSA and pSB, FIG. 1). This allowed the resulting PCR product to be digested with the Sfi1 enzyme, yielding different overhang sequences on the amplified DNA. The amplified coding sequence was kept in frame with other translational signals such as the initiator ATG during primer design.

1B. PCR Amplification of Protein-Encoding DNA

In this step, the desired region of a gene was amplified. The amplification production can be the entire ORF (open reading frame) of a gene or a selected part of it. Use of the partial ORF will lead to epitope(or domain)-specific immune responses upon immunization.

The template for the PCR can be either RNA (via RT-PCR) or cDNA. Conventional PCR parameters are known to those of skill in the art and work well. An example is a cycling scheme of [(58C-30 sec), (72C-1 to 3 min), (94C-20 sec)] with a total of 25 cycles.

1C. Digestion of PCR-amplified DNA

Sfi1 enzyme was added directly into the PCR reactions for digestion for 1 hour to create Sfi1 ends on the DNA. As Sfi1 cut leaves 5′ overhangs rather than 3′ overhangs, there was no need for filling-in reactions to form blunt ends while enzyme digestion proceeded during the PCR reaction. After restriction, DNA is purified using any conventional method, well known in the art.

1D. Production of Promoter and polyA Fragments

A plasmid was engineered to contain the following elements in 5′ to 3′ order: the CMV promoter, the Kozak translation signal sequence with initiator ATG, Ig-kappa secretion signal sequence, Sfi1A, a stuffer sequence, stop codon (TAG), Sfi1B, and the SV40 polyA sequence. The plasmid DNA was prepared and digested with Sfi to remove the stuffer sequence. The vector fragment, with Sfi ends, was then purified by agarose electrophoresis and was ligated to the protein-encoding DNA as prepared in Example 1C.

1E. Ligation of Digested DNA to a Promoter and a polyA

The coding DNA fragment prepared in Example 1C and the vector fragment prepared in Example 1D were ligated with a DNA ligase. Depending on the type of ligase kit, the ligation can be complete in minutes or, in the case of conventional ligation, in several hours, or overnight. A typical ligation is a 20 microliter reaction with a 5:1 molar ratio of encoding DNA (1C): vector DNA (1D) with a total DNA of 0.1-0.2 microgram.

1F. Final PCR Amplification

An aliquot of the ligation mixture was used directly as template for PCR amplification of the expression competent DNA fragment. Typically, 1 microliter is used for a 50 microliter PCR and use of more ligation mixture than 1 μl did not help increase the final yield. The yield from an amplification PCR of 50 microliters usually ranges from 2 to 8 micrograms. The remaining ligation mixture can be retained for, if necessary, later transformation of bacteria for plasmid DNA.

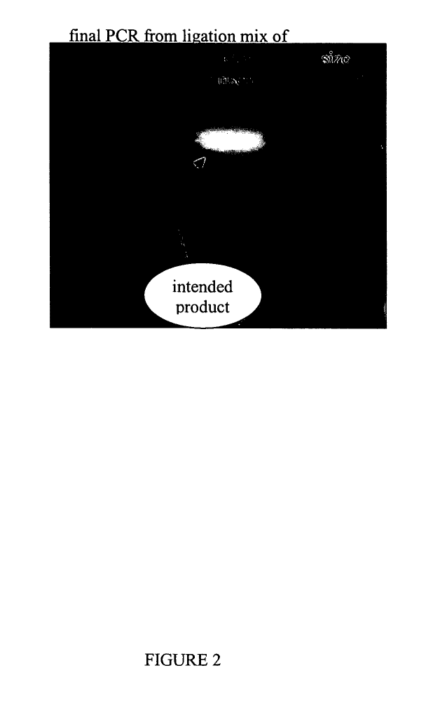

An example of the final amplified products is shown in FIG. 2. The C-terminal 300 bp open reading frame sequence of human MTF was isolated by RT-PCR from a universal RNA preparation (Invitrogen) using Sfi1-containing primers. The PCR reaction was digested with Sfi and the DNA was purified with a spin-column (Qiagen). The purified DNA was ligated to the engineered Sfi1 vector overnight, with vector alone ligation as negative control. 1 microliter aliquots of the ligation reactions were used for the second (final) round of PCR to add the sequences of the CMV promoter and the polyA sequence. A final yield of 7 microgram DNA was obtained from a 50 microliter PCR with the 1 microliter of the ligation reaction as template, while the control PCR (using the ligation of vector DNA alone as template) produced faint nonspecific bands.

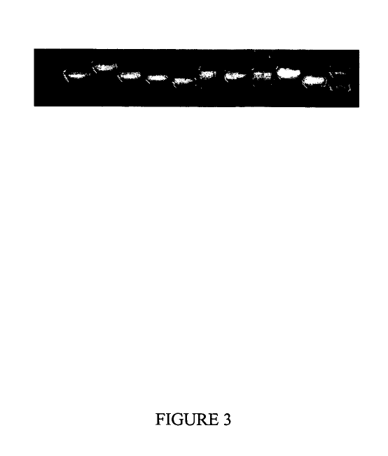

Additionally, expression-competent DNA fragments for 10 genes were successfully batch-produced using the above methods. See, FIG. 3.

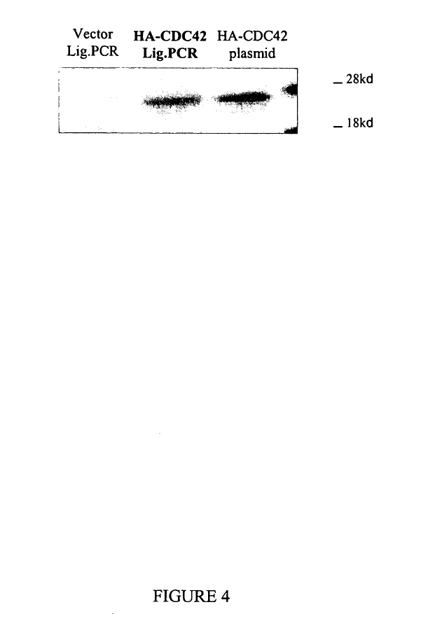

To confirm that the DNA products from the ligation-assisted amplification were capable of expressing the desired protein, the expression-competent DNA for a test gene, CDC42 (human), was produced (expected to encode HA-tagged CDC42 protein) and transfected, using Invitrogen's lipofectamine kit, into HEK293 cells (ATCC Accession NO. CRL-1573). 40 hrs post-transfection, cells were lysed and lysates subjected to SDS-PAGE and immunoblotting with anti-HA antibody (Babco, Richmond, Calif.). The PCR product from the ligation PCR, as well as the positive control plasmid), but not that from the vector-alone ligation, clearly produced the expected protein (HA-CDC42). See, FIG. 4.

### EXAMPLE 2

### Production of Plasmid Adjuvant

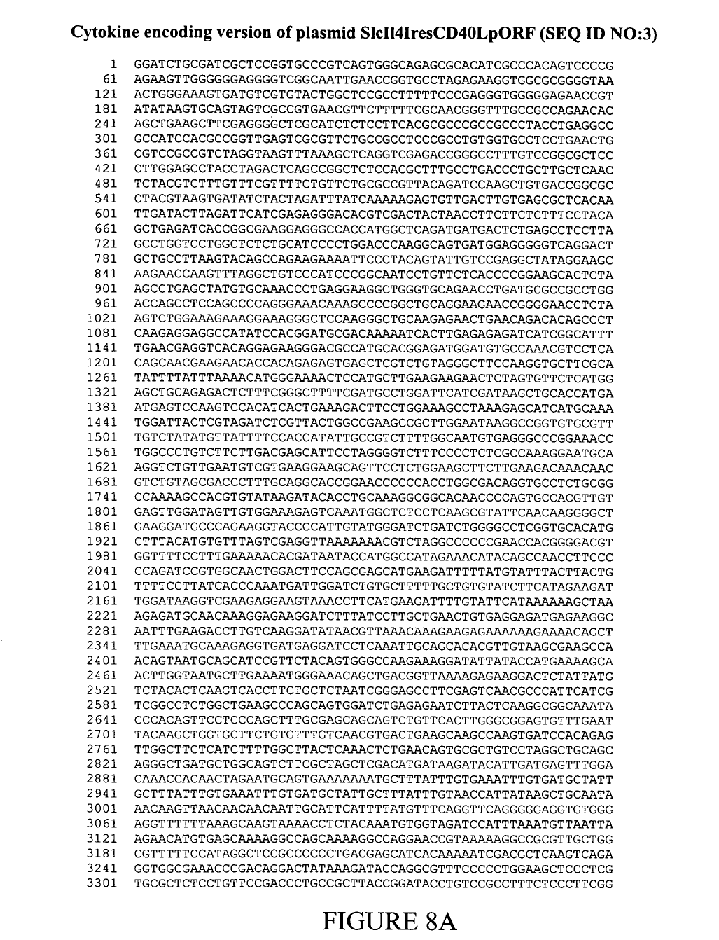

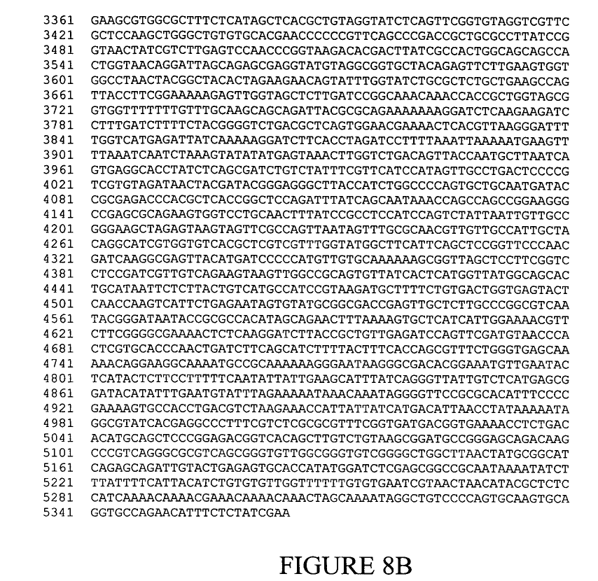

A bacterial plasmid construct was produced for use as an adjuvant for co-electroporation. The plasmid, named SlcIl4IresCD40LpORF (SEQ ID NO:3), has the configuration of EF1alpha-SLC/IL4 fusion-IRES-CD40 ligand as depicted in FIG. 5 and FIGS. 8A-B. The plasmid was produced by PCR linking the different fragments into pORF-mCD40L v.15 (InvivoGen, San Diego, Calif.). This plasmid contains the CD40 ligand sequence. The IL4 sequence was from pORF-mIL04 v.11 (InvivoGen). The SLC and IRES sequences were from pGT60mExodus2 v.02 (InvivoGen).

### EXAMPLE 3

### Immunization of Animals Via In Vivo Electroporation

Animal immunization was achieved electrically through the electroporation of leg tissues with the antigen-encoding DNA. Briefly, the TA (tibialis anterior) muscle regions of the two hind legs were shaved and 50 μl of the DNA fragments (5-15 microgram) in PBS (phosphate-buffered saline) were injected into each muscle. Using an electroporator from BTX Molecular Delivery Systems (ECM 830), electric shocks were delivered as: 100 volts; pulse length of 50 milliseconds, 200 millisecond pulse interval, 5 pulses total. Boost electroporation, performed identical to the primary immunization, was carried out 2 weeks after the primary. The mice were sacrificed 4 weeks later for antisera for analyses.

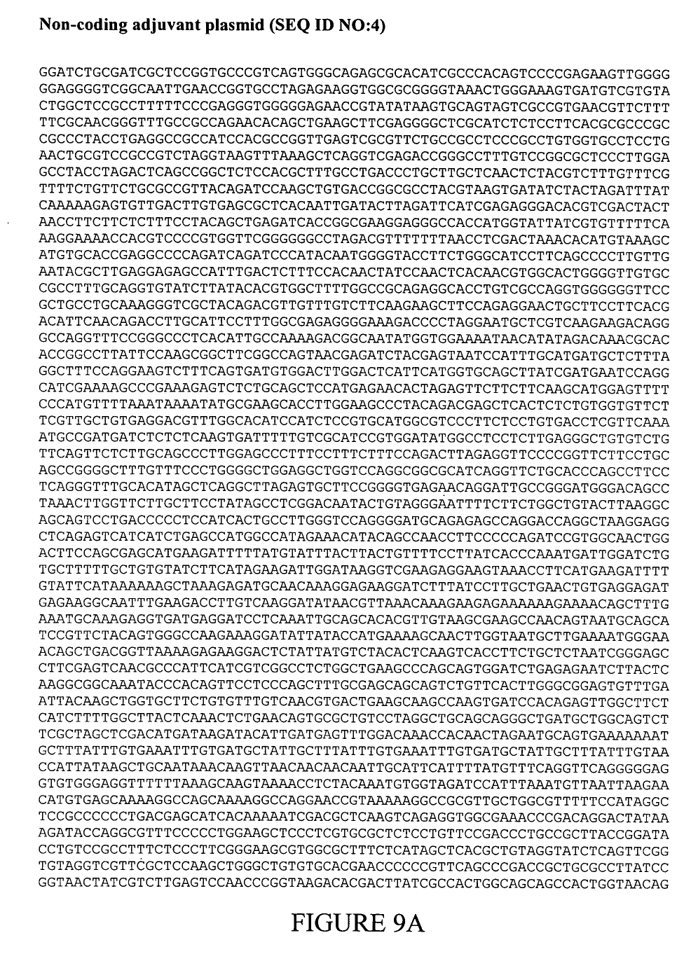

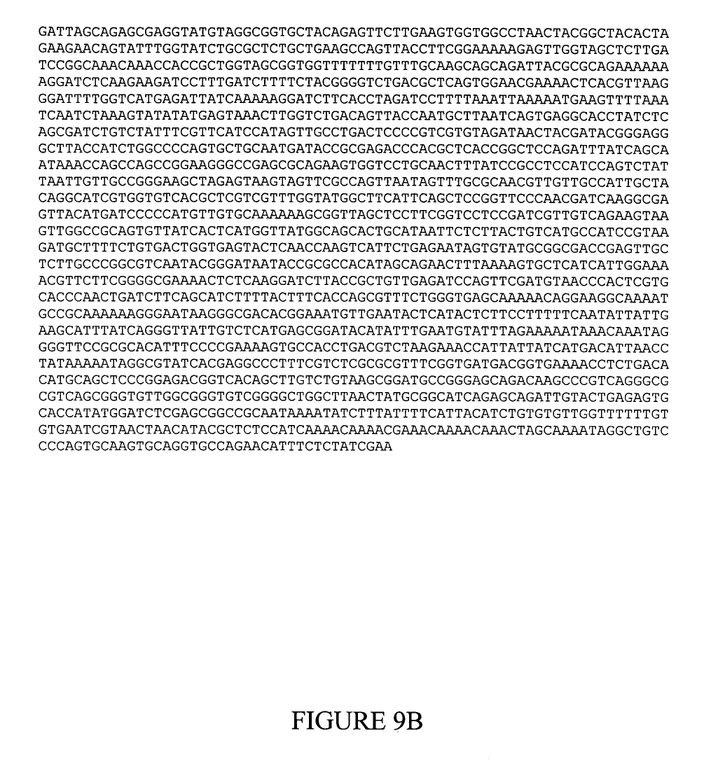

Five groups of Balb/c mice were also immunized via electroporation with 5 micrograms PSA (human prostate specific antigen)-expressing DNA fragments (prepared according to the procedures above) along with adjuvant plasmid or its non-coding variant (15 microgram each) and boosted at 2 weeks. 4 weeks later the antisera were diluted 1:2000 and analyzed by ELISA for anti-PSA antibody titers. The non-coding variant (SEQ ID NO:4, FIGS. 9A-B) contained the cytokine segment in the opposite orientation and was therefore not expected to encode the cytokines.

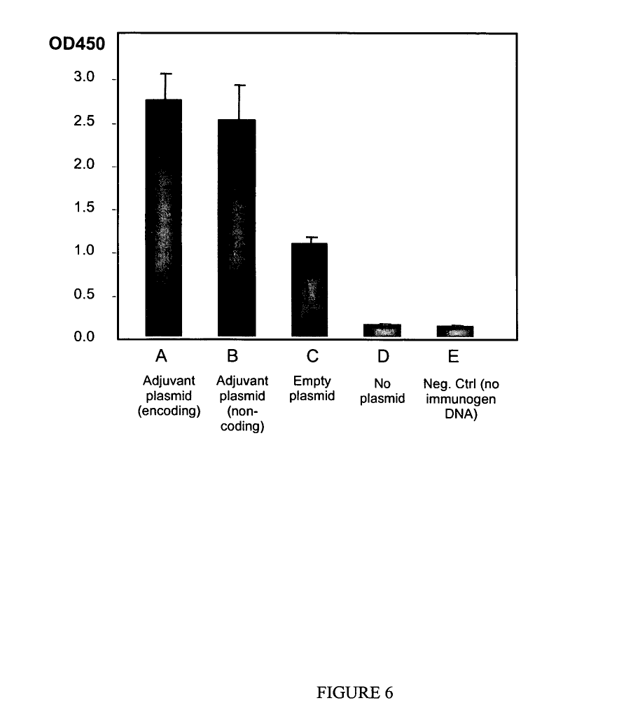

Results are shown in FIG. 6. A, B, C, D and E shown on the horizontal axis are as follows: (A) animals administered adjuvant plasmid (encoding), containing a cytokine-encoding segment consisting of SLC-IL4 fusion, IRES and CD40 ligand under the transcriptional control of the EF1alpha promoter, and is expected to encoding the cytokine proteins; (B) animals administered its non-coding variant that contains the cytokine segment in the opposite orientation and is therefore not expected to encode the cytokines; (C) animals given an empty plasmid that lacks the promoter and the cytokine segment; (D) animals given PSA without any plasmid DNA; and (E) animals that were immunized with a negative control containing no immunogen.

As can be seen, a comparison of groups A and D indicates that the adjuvant plasmid was highly effective in promoting antibody production (some 15 folds). A comparison of groups A and B indicates a marginal but detectable effect of the cytokine proteins. A comparison of groups B and C suggests that sequences of EF1alpha promoter, SLC-IL4, IRES and/or CD40 ligand may be important in enhancing antibody response.

### EXAMPLE 4

### Effect of a Signal Sequence on the Antibody Response

Expression of intracellular proteins does not always vigorously activate an antibody response because their intracellular location can inhibit their recognition by antigen-presenting cells. In order to test whether the use of a secretory signal enhanced the antibody response to DNA fragments produced under the invention, the following experiment was conducted.

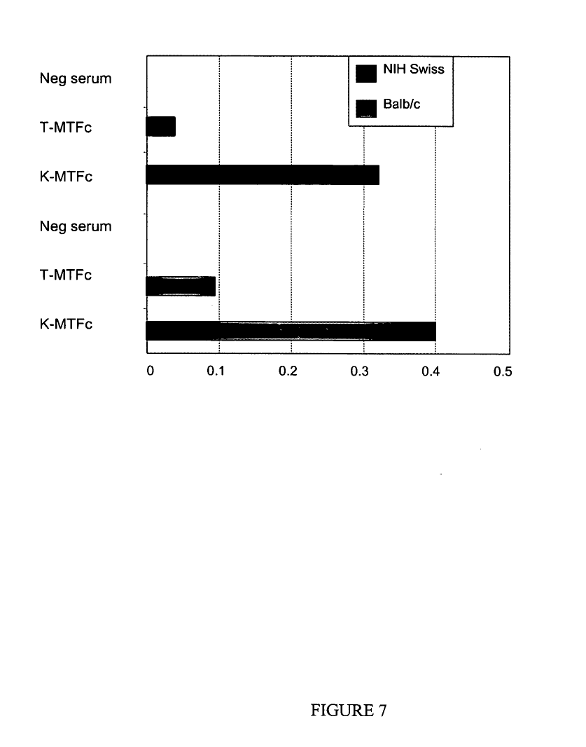

Two signal sequences capable of translocating molecules outside of the cell were tested: a TAT sequence (11 amino acids) of the HIV sequence, and the Ig-kappa secretory sequence (21 amino acids). A 1.2 kb cDNA encoding the C-terminus of human MTF was isolated by RT-PCR and tagged by one of the two sequences by PCR. Expression-competent DNA fragments were produced and used to immunize mice (Balb/c and NIH Swiss) according the procedures described above. Adjuvant plasmid DNA as described above was included in equal quantity to the immunogen DNA. The antisera were analyzed with bead-immobilized purified GST-MTFc using an ELISA assay. As shown in FIG. 7, antibody production was elicted using both signal sequences but the kappa sequence showed greatly enhanced responses as compared to the TAT sequence.

Thus, methods for producing and using immunogen DNA through amplification methodology including ligation-assisted PCR are described. Also described is an adjuvant plasmid to enhance antibody production. Although preferred embodiments of the subject invention have been described in some detail, it is understood that obvious variations can be made without departing from the spirit and the scope of the invention as defined herein.

1. A high-throughput method of preparing an immunogenic vector, comprising:
a) amplifying a polynucleotide sequence encoding an immunogenic polypeptide or an immunogenic fragment thereof, using a forward primer, comprising a first restriction site for an infrequently cutting restriction endonuclease, and a reverse primer, comprising a second restriction site for an infrequently cutting restriction endonuclease, to obtain an amplified polynucleotide product containing said first and second restriction sites flanking said polynucleotide sequence;
b) digesting the amplified polynucleotide with said infrequently cutting restriction endonuclease;
c) ligating digested amplified polynucleotide into a vector cut with the same infrequently cutting restriction endonuclease used to digest said amplified polynucleotide, wherein said vector contains at least one control element compatible with expression of said immunogenic polypeptide or immunogenic fragment thereof in a host cell; and
d) amplifying said vector, comprising the inserted polynucleotide encoding said immunogenic polypeptide or immunogenic fragment thereof.

2. The method of claim 1, wherein the vector, the amplified polynucleotide is ligated into, comprises a stuffer sequence flanked on both sides by the same or different restriction sites for an infrequently cutting restriction endonuclease.

3. The method of claim 2, wherein the vector, the amplified polynucleotide is ligated into, comprises in 5′ to 3′ order:
a) a CMV promoter,
b) a Kozak translation signal,
c) an ATG start codon,
d) an Ig-kappa secretion signal,
e) a first Sfi1 endonuclease restriction site,
f) a stuffer sequence that can be removed by digestion with Sfi1 endonuclease,
g) a TAG stop codon,
h) a second Sfi1 endonuclease restriction site, and
i) an SV40 polyadenylation sequence.

4. The method of claim 1, wherein the polynucleotide encoding an immunogenic polypeptide or immunogenic fragment thereof is derived from an organism selected from the group consisting of:
a) a bacteria,
b) a virus,
c) a fungus, and
d) a parasite.

5. The method of claim 1, wherein the polynucleotide encoding an immunogenic polypeptide or immunogenic fragment thereof further comprises a sequence encoding a signal peptide.

6. The method of claim 5, wherein the signal peptide is selected from the group consisting of:
a) a yeast invertase signal peptide,
b) an α-factor signal peptide,
c) an interferon signal peptide,
d) an adenovirus tripartite signal peptide,
e) a tpa signal peptide,
f) a tat signal peptide, and
g) an Ig-kappa signal peptide.

7. The method of claim 1, wherein the forward primer comprises the sequence of SEQ ID NO: 1 and the reverse primer comprises the sequence of SEQ ID NO:2.

8. The method of claim 1, wherein the method of amplifying the polynucleotide, encoding an immunogenic polypeptide or an immunogenic fragment thereof, is selected from the group consisting of:
a) PCR,
b) RTPCR,
c) LCR,
d) NASBA,
e) Q-beta amplification,
f) strand displacement amplification, and
g) target mediated amplification.

9. The method of claim 1, wherein the immunogenic vector produced by the method of claim 1, comprises said polynucleotide sequence, encoding an immunogenic polypeptide or an immunogenic fragment thereof, operably linked to at least one control element compatible with expression in a vertebrate host cell.

10. The method of claim 9, wherein the immunogenic vector comprises a control element selected from the group consisting of a transcription promoter, a transcription enhancer element, a transcription termination signal, a UTR sequence, a polyadenylation sequence, a sequence for optimization of initiation of translation, and a translation termination sequence.

11. The method of claim 10, wherein the immunogenic vector comprises a promoter selected from the group consisting of:
a) an SV40 promoter,
b) a CMV promoter,
c) a mouse mammary tumor virus LTR promoter,
d) an adenovirus major late promoter,
e) a herpes simplex virus promoter,
f) an EF1alpha promoter, and
g) a promoter derived from the murine metallothionein gene.

12. The method of claim 10, wherein the immunogenic vector comprises a transcription enhancer element selected from the group consisting of:
a) an SV40 enhancer element,
b) a LTR derived enhancer element,
c) a Rous Sarcoma Virus enhancer element, and
d) a CMV enhancer element.

13. The method of claim 10, wherein the immunogenic vector comprises a transcription termination signal selected from the group consisting of:
a) an SV40 transcription termination signal, and
b) a bovine growth hormone transcription termination signal.

14. The method of claim 10, wherein the immunogenic vector comprises an internal ribosome entry site (IRES) sequence.

15. The method of claim 10, wherein said vector, comprising the polynucleotide encoding an immunogenic polypeptide or an immunogenic fragment thereof, is amplified to produce immunizing DNA by a method selected from the group consisting of:
a) PCR,
b) RTPCR,
c) LCR,
d) NASBA,
e) Q-beta amplification,
f) strand displacement amplification, and
g) target mediated amplification.

16. A composition comprising the immunizing DNA produced by the method of claim 15.

17. The composition of claim 16, further comprising an adjuvant.

18. The composition of claim 16, further comprising a pharmaceutically acceptable excipient.

19. A method of immunization of a subject, the method comprising, introducing the composition of claim 16 into said subject under conditions that are compatible with expression of the polynucleotide, encoding an immunogenic polypeptide or immunogenic fragment thereof, in said subject.

20. A method of generating an immune response in a subject, comprising: providing the immunizing DNA of claim 16,
expressing said immunogenic polypeptide or immunogenic fragment thereof in a suitable host cell,
isolating said immunogenic polypeptide or immunogenic fragment thereof, and administering said immunogenic polypeptide or immunogenic fragment thereof to the subject in an amount sufficient to elicit an immune response.

21. A method of generating an immune response in a subject, comprising introducing into cells of said subject the immunizing DNA of claim 16, under conditions that permit the expression of said polynucleotide and production of said immunogenic polypeptide or immunogenic fragment thereof, thereby eliciting an immunological response to said immunogenic polypeptide or immunogenic fragment thereof.

22. The method of claim 21, further comprising introducing into cells of said subject an adjuvant plasmid comprising the sequence of SEQ ID NO:3, under conditions that permit the expression of said cytokine-encoding segment.

23. The method of claim 21, further comprising introducing into cells of said subject the non-coding adjuvant plasmid comprising the sequence of SEQ ID NO:4.

24. A method of making a polyclonal antibody, the method comprising:
a) introducing the immunizing DNA of claim 16 into an animal under conditions that permit the expression of said polynucleotide and production of said immunogenic polypeptide or an immunogenic fragment thereof, thereby eliciting an antibody response in said animal,
b) isolating antibodies from the animal, and
c) screening the isolated antibodies with said immunogenic polypeptide or an immunogenic fragment thereof, thereby identifying a polyclonal antibody which specifically binds to said immunogenic polypeptide.

25. A method of making a polyclonal antibody, the method comprising:
a) introducing the immunizing DNA of claim 16 into a suitable host cell under conditions that permit the expression of said polynucleotide and production of said immunogenic polypeptide or immunogenic fragment thereof,
b) isolating said immunogenic polypeptide or immunogenic fragment thereof,
c) immunizing an animal with the immunogenic polypeptide or immunogenic fragment thereof under conditions to elicit an antibody response,
d) isolating antibodies from the animal, and
e) screening the isolated antibodies with said polypeptide, thereby identifying a polyclonal antibody which specifically binds to said polypeptide.

26. A method of making a monoclonal antibody, the method comprising:
a) introducing the immunizing DNA of claim 16 into an animal under conditions that permit the expression of said polynucleotide and production of said immunogenic polypeptide or an immunogenic fragment thereof, thereby eliciting an antibody response in said animal,
b) isolating antibody producing cells from the animal,
c) fusing the antibody producing cells with immortalized cells to form monoclonal antibody-producing hybridoma cells,
d) culturing the hybridoma cells, and
e) isolating from the culture a monoclonal antibody which specifically binds to said immunogenic polypeptide.

27. A method of making a monoclonal antibody, the method comprising:
a) introducing the immunizing DNA of claim 16 into a suitable host cell under conditions that permit the expression of said polynucleotide and production of said immunogenic polypeptide or immunogenic fragment thereof,
b) isolating said immunogenic polypeptide or immunogenic fragment thereof,
c) immunizing an animal with the immunogenic polypeptide or immunogenic fragment thereof under conditions to elicit an antibody response,
d) isolating antibody producing cells from the animal,
e) fusing the antibody producing cells with immortalized cells to form monoclonal antibody-producing hybridoma cells,
f) culturing the hybridoma cells, and
g) isolating from the culture a monoclonal antibody which specifically binds to said immunogenic polypeptide.

28. A method of generating a phage display library, the method comprisng:
a) introducing the immunizing DNA of claim 16 into a suitable host cell under conditions that permit the expression of said polynucleotide and production of said immunogenic polypeptide or immunogenic fragment thereof,
b) isolating said immunogenic polypeptide or immunogenic fragment thereof,
(c) providing a library of filamentous bacteriophage, each filamentous bacteriophage displaying at its surface an antibody molecule, and each filamentous bacteriophage containing nucleic acid encoding a polypeptide chain which is a component part of the antibody molecule displaying at the surface of that filamentous bacteriophage;
(d) selecting from said library of filamentous phage by binding with said immunogenic polypeptide or an immunogenic fragment thereof, one or more displayed antibody molecules having binding specificity for said immunogenic polypeptide.


SEQUENCE LISTING (4 sequences)

SEQ ID NO: 1 - DNA, 13 bp, Artificial
  PCR primer pSA
ggccnnnnnggcc

SEQ ID NO: 2 - DNA, 13 bp, Artificial
  PCR primer pSB
ggccnnnnnggcc

SEQ ID NO: 3 - DNA, 5365 bp, Artificial
  Cytokine encoding version of plasmid SlcIl4IresCD40LpORF
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggctcagatgatgactctgagcctccttagcctggtcctggctctctgcatcccctggacccaaggcagtgatggagggggtcaggactgctgccttaagtacagccagaagaaaattccctacagtattgtccgaggctataggaagcaagaaccaagtttaggctgtcccatcccggcaatcctgttctcaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagaggaggccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtagatctcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggccatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctgaacagtgcgctgtcctaggctgcagcagggctgatgctggcagtcttcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 4 - DNA, 5365 bp, Artificial
  Non-coding adjuvant plasmid
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggtattatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgagatctacgagtaatccatttgcatgatgctctttaggctttccaggaagtctttcagtgatgtggacttggactcattcatggtgcagcttatcgatgaatccaggcatcgaaaagcccgaaagagtctctgcagctccatgagaacactagagttcttcttcaagcatggagttttcccatgttttaaataaaatatgcgaagcaccttggaagccctacagacgagctcactctctgtggtgttcttcgttgctgtgaggacgtttggcacatccatctccgtgcatggcgtcccttctcctgtgacctcgttcaaaatgccgatgatctctctcaagtgatttttgtcgcatccgtggatatggcctcctcttgagggctgtgtctgttcagttctcttgcagcccttggagccctttcctttctttccagacttagaggttccccggttcttcctgcagccggggctttgtttccctggggctggaggctggtccaggcggcgcatcaggttctgcacccagccttcctcagggtttgcacatagctcaggcttagagtgcttccggggtgagaacaggattgccgggatgggacagcctaaacttggttcttgcttcctatagcctcggacaatactgtagggaattttcttctggctgtacttaaggcagcagtcctgaccccctccatcactgccttgggtccaggggatgcagagagccaggaccaggctaaggaggctcagagtcatcatctgagccatggccatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctgaacagtgcgctgtcctaggctgcagcagggctgatgctggcagtcttcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa